(19) United States
(12) Patent Application Publication
(10) Pub. No.: US 2014/0307756 A1
(43) Pub. Date: Oct. 16, 2014

(54) APPARATUS AND METHOD FOR MEASURING CALORIE IN A BEVERAGE

(71) Applicant: KONINKLIJKE PHILIPS N.V., EINDHOVEN (NL)

(72) Inventors: Weizhong Chen, Shanghai (CN); Bin Yin, Shanghai (CN); Declan Patrick Kelly, Shanghai (CN)

(21) Appl. No.: 14/355,681

(22) PCT Filed: Nov. 2, 2012
(86) PCT No.: PCT/IB2012/056126

§ 371 Date: May 1, 2014

(30) Foreign Application Priority Data
Nov. 3, 2011 (CN) PCT/CN2011/081728

(51) Int. Cl. G01N 33/14; G01K 17/02

(52) U.S. Cl. CPC G01N 33/14; G01K 17/02
USPC 374/31

(57) ABSTRACT

The invention relates to an apparatus and method for measuring calorie in a beverage. The apparatus comprises a chamber, an information obtaining unit, a concentration measuring unit, a processing unit and a display screen. The chamber is configured to contain the beverage. The information obtaining unit is configured to obtain beverage information indicating the volume or the weight of the beverage. The concentration measuring unit configured to measure the concentration of a predetermined substance in the beverage. The processing unit is configured to calculate the overall calorie according to the volume or the weight of the beverage and the measured concentration of the predetermined substance. The display screen is configured to display the overall calorie.

### FIELD OF THE INVENTION

The invention generally relates to measurement technology, and more particularly, to an apparatus and method for measuring calorie in a beverage.

### BACKGROUND OF THE INVENTION

Obesity is regarded as one of the most serious public health problems in the 21^{st} century. One factor contributing to the obesity epidemic is increasing dietary energy intake from beverages. Many studies suggest that an increase in consumption of beverages containing high calorie results in weight gain. Therefore, for the over-weighted population or the population with certain illnesses such as diabetes, monitoring their calorie intake, especially sugar intake, will be crucial to their health. In addition, for those people who need to monitor their calorie intake, a convenient device to measure the calorie in their beverage is also important.

Generally the calorie contained in a food or beverage is measured by a “bomb calorimeter”, which comprises a solid metal container surrounded by a water bath. To measure the calorie in the food or the beverage, a test sample of the food or the beverage is dehydrated and ground into powder. Then the test sample powder is placed into the calorimeter filled with pure oxygen. The test sample powder is ignited and explodes in the calorimeter. As a result, the calorie, i.e. the chemical energy contained in the test sample will be transferred into heat by the explosion, which increases the temperature within the calorimeter. In this way, the calorie contained in the food or the beverage can be accurately measured. However, this calorimeter is complicated and inconvenient to use in daily life.

### OBJECT AND SUMMARY OF THE INVENTION

It would be, therefore, advantageous to achieve an apparatus and method capable of measuring the overall calorie contained in a beverage.

To this end, in one aspect of the invention, there is provided an apparatus for measuring calorie in a beverage, which comprises: a chamber configured to contain the beverage; an information obtaining unit configured to obtain beverage information indicating the volume or the weight of the beverage; a concentration measuring unit configured to measure the concentration of a predetermined substance in the beverage; a processing unit configured to calculate the overall calorie according to the volume or the weight of the beverage and the measured concentration of the predetermined substance; and a display screen configured to display the overall calorie.

With the concentration measuring unit, the concentration of the predetermined substance contributing to the overall calorie in the beverage can be accurately measured, which helps to determine the weight of the predetermined substance. In this way, the overall calorie in the beverage can be measured and then displayed to users. Moreover, this apparatus can be integrated into a cup, a water supplier or other liquid containing devices. Thus, it is more convenient for users to estimate and monitor their calorie intake from beverages with such apparatus.

In an embodiment, the information obtaining unit is a first sensor for measuring the volume or the weight of the beverage, or a first input unit for receiving an instruction including the beverage information.

In an embodiment, the concentration measuring unit further comprises: a second sensor, configured to measure the propagation characteristic of an ultrasonic wave passing through the beverage; a thermometer, configured to measure the temperature of the beverage; and the concentration measuring unit is further configured to determine the concentration of the predetermined substance according to the temperature of the beverage and the propagation characteristic of the ultrasonic wave. Since the propagation characteristic of the ultrasonic wave, such as the velocity, the time of flight or the amplitude attenuation, is significantly dependent on the concentration of the beverage in which the ultrasonic wave propagates, this propagation characteristic can be used to determine the concentration of the beverage.

In an embodiment, the apparatus further comprises: a second input unit, configured to receive a user instruction including the type of the predetermined substance and/or the type of the beverage; and the processing unit is further configured to calculate the overall calorie according to the type of the predetermined substance and/or the type of the beverage. In this way, the users can input the user instruction indicating the type of the beverage and/or the type of the predetermined substance to the apparatus by the second input unit, which enables the apparatus to identify different calorie-contributing substances, thereby improving the accuracy of the calorie measurement.

In an embodiment, the apparatus further comprises: a memory, configured to store the results of the overall calorie; and the apparatus is further configured to provide historical calorie intake information according to the results of the overall calorie. The historical calorie intake information can be used as the basis of health advices or drink recommendations for users.

In another aspect of the invention, there is provided a method for measuring calorie in a beverage, which comprises the steps of: obtaining beverage information indicating the volume or the weight of the beverage; measuring the concentration of a predetermined substance in the beverage; calculating the overall calorie according to the volume or the weight of the beverage and the measured concentration of the predetermined substance; and displaying the overall calorie.

Detailed explanations and other aspects of the invention will be given below.

### BRIEF DESCRIPTION OF THE DRAWINGS

The particular aspects of the invention will now be explained with reference to the embodiments described hereinafter and considered in connection with the accompanying drawings, in which identical parts or sub-steps are designated in the same manner:

FIG. 1 depicts an apparatus 100 for measuring calorie according to a first embodiment of the invention;

FIG. 2 depicts an apparatus 200 for measuring calorie according to a second embodiment of the invention;

FIG. 3 depicts a method 300 for measuring calorie according to a third embodiment of the invention; and

FIG. 4 depicts a method 400 for measuring calorie according to a fourth embodiment of the invention.

### DETAILED DESCRIPTION OF THE INVENTION

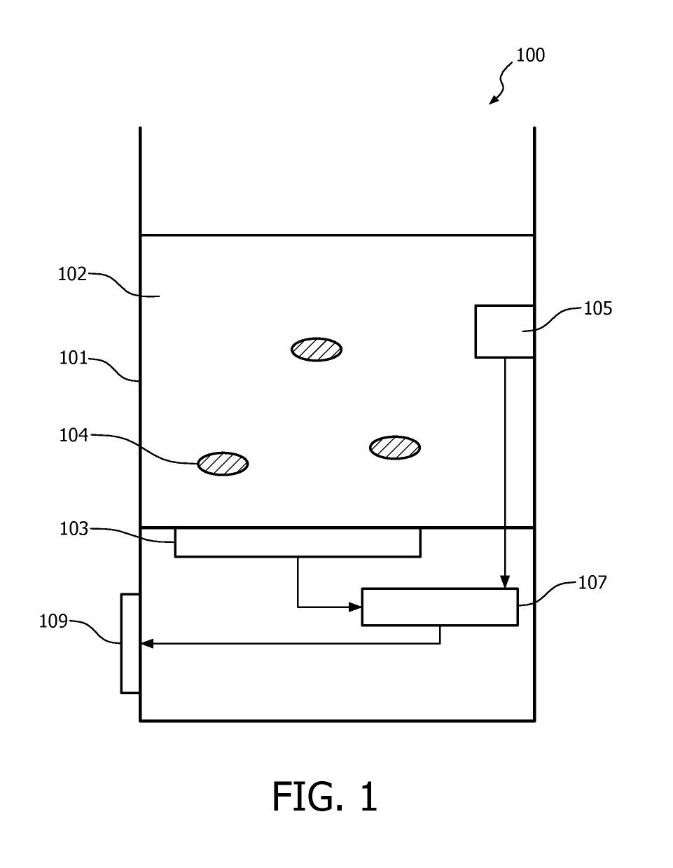

FIG. 1 depicts an apparatus 100 for measuring calorie in a beverage according to a first embodiment of the invention. In some embodiments, the apparatus 100 can be integrated into a beverage container, such as a cup, a bottle, a kettle or any other suitable vessels. In some other embodiments, the apparatus 100 can be integrated into a beverage making machine or a beverage supplying machine, such as a carbonated drink machine or a juicer, which is adapted to dispense the beverage through a supply channel.

As shown in FIG. 1, the apparatus 100 comprises:
a chamber 101, configured to contain the beverage 102;
an information obtaining unit 103, configured to obtain beverage information indicating the volume or the weight of the beverage 102;
a concentration measuring unit 105, configured to measure the concentration of a predetermined substance 104 in the beverage 102;
a processing unit 107, configured to calculate the overall calorie according to the volume or the weight of the beverage 102 and the measured concentration of the predetermined substance; and
a display screen 109, configured to display the overall calorie.

For example, the beverage 102 may correspond to tea drinks, fruit juices, energy drinks, carbonated drinks, alcoholic beverage or any other beverages containing one or more calorie-contributing substances. The beverage 102 has a roughly uniform concentration. For example, the predetermined substance 104 may comprise one or more of sugar, alcohol, protein, fat or any other substance that contributes to the overall calorie in the beverage 102.

In the embodiment, the information obtaining unit 103 is a first sensor for measuring the volume or the weight of the beverage 102. The first sensor can be disposed on the bottom of the chamber 101, or disposed along a wall of the chamber 101. For example, the first sensor is a weight sensor, which is configured to measure the weight of the beverage 102. In some other examples, the first sensor is a level meter or a flow meter, which is configured to measure the volume of the beverage 102. The information obtaining unit 103 is electrically connected to the processing unit 107. Then a first signal including the beverage information is delivered from the information obtaining unit 103 to the processing unit 107.

The concentration measuring unit 105 can be disposed inside the chamber 101. Alternatively, the concentration measuring unit 105 can be arranged on the wall of the chamber 101, for example, mounted on the inner wall or outer wall of the chamber 101. In the embodiment, the concentration measuring unit 105 utilizes the propagation characteristic of an ultrasonic wave to determine the concentration of the predetermined substance 104, as the concentration of the predetermined substance 104 in which the ultrasonic wave propagates significantly influences the propagation characteristic of the ultrasonic wave, such as the velocity, the time of flight or the amplitude attenuation. Specifically, the concentration measuring unit 105 comprises a second sensor (not shown) and a thermometer (not shown). The second sensor is configured to measure the propagation characteristic of the ultrasonic wave passing through the beverage 102. The thermometer is configured to measure the temperature of the beverage 102. The measured propagation characteristic of the ultrasonic wave and the temperature of the beverage 102 can be delivered to the processing unit 107 or another signal processing unit (not shown) to determine the concentration of the predetermined substance 104. The processing unit 107 is provided with a look-up table, a calibration curve or the like, which interprets the relationship between the concentration of the predetermined substance 104 and the propagation characteristic of the ultrasonic wave under various temperatures. The relationship between the concentration of the predetermined substance 104 and the propagation characteristic of the ultrasonic wave can be pre-determined according to a series of measurements for beverages of different concentrations and temperatures. In this way, the concentration of the predetermined substance 104 can be accurately measured.

In some other examples, the concentration measuring unit 105 is a refractometer for measuring the index of refraction of the beverage 102. The refractometer generally comprises a light source (not shown) and a photo detector (not shown) being disposed on the wall of the chamber 101. The index of refraction can be determined based on the critical angle of refraction in the beverage 102. The concentration of the predetermined substance 104 in the beverage 102 is generally associated with the index of refraction of the beverage 102. In this way, the concentration of the predetermined substance 104 can be accurately measured according to the index of refraction. It is readily appreciated that the concentration measuring unit 105 may be other suitable measuring devices capable of measuring the concentration of the predetermined substance 104.

The concentration measuring unit 105 is electrically connected to the processing unit 107. A second signal reflecting the concentration of the predetermined substance 104 is delivered to the processing unit 107. The processing unit 107 may be implemented by hardware, software, firmware and/or any combination of hardware, software and/or firmware. For example, the processing unit 107 may be implemented by one or more circuit(s), programmable processor(s), ASIC(s), PLD(s), FPGA(s) or any other suitable devices. Upon receiving the first and the second signals, the processing unit 107 calculates the overall calorie according to the volume or the weight of the beverage 102 and the measured concentration of the predetermined substance 104. Specifically, the processing unit 107 calculates the mass of the predetermined substance 104 contained in the beverage 102 by multiplying the concentration of the predetermined substance 104 and the volume of the beverage 102. The volume of the beverage 102 can be directly measured, or determined according to the weight of the beverage 102 and the density of the beverage 102. Then the overall calorie can be calculated according to the mass of the predetermined substance 104 and the calorific value of the predetermined substance 104. In some other examples, the mass of the predetermined substance 104 contained in the beverage 102 can also be calculated by multiplying the weight of the beverage 102 with the concentration of the predetermined substance 104, which is in the form of mass percent concentration or the like. The overall calorie can be displayed by the display screen 109, which can be disposed on the outer surface of the chamber 101 or some other locations, like the lid. For example, the display screen 109 is an LED screen. In some examples, the display screen 109 may display other relevant parameters such as the temperature of the beverage or the weight/volume of the beverage.

In some examples, the apparatus 100 may further comprise a memory (not shown), which is configured to store the historical results of the overall calorie. Thus, the apparatus 100 can be used to provide historical calorie intake information according to the results of the overall calorie stored in the memory. For example, the historical calorie intake information includes how much calorie the user has taken from by the apparatus 100 in a selected past period. Moreover, the memory can be used to store user profiles which include the BMI (Body Mass Index), age, gender, diet, weight information associated with the users. The processing unit 107 can generate a target value or recommendatory value based on the user profiles. Then the processing unit 107 can compare the historical calorie intake information with the target value or recommendatory value to provide health advices or drink recommendations on future calorie intake for the users.

Moreover, since the components within the apparatus 100 are generally small, the apparatus 100 can be formed as a portable device with compact design and light weight. The compact apparatus 100 is more convenient for the user, and also much easier to measure the calorie in the beverages.

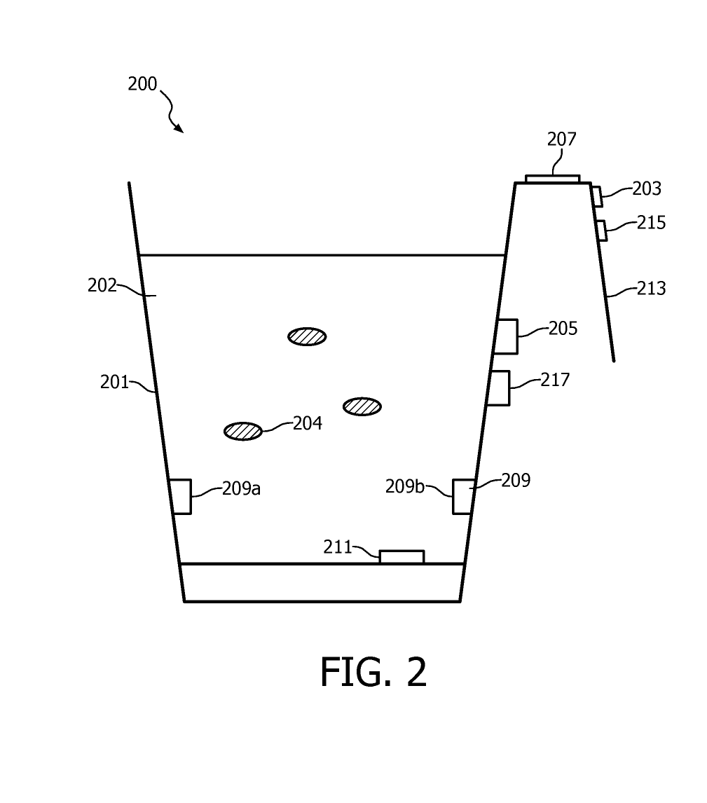

FIG. 2 depicts an apparatus 200 for measuring calorie according to a second embodiment of the invention. As shown in FIG. 2, the apparatus 200 comprises a chamber 201, an information obtaining unit 203, a concentration measuring unit, a processing unit 205 and a display screen 207. It is readily appreciated that most of the components within the apparatus 200 are electrically connected via wires (not shown) to deliver signals therebetween.

In the embodiment, the information obtaining unit 203 is a first input unit, which is configured to receive an instruction including the beverage information. For example, the instruction can be input by the user. The concentration measuring unit comprises a second sensor 209 for measuring the propagation characteristic of an ultrasonic wave passing through the beverage 202, and a thermometer 211 for measuring the temperature of the beverage 202. The second sensor 209 comprises a first portion 209a and a second portion 209b disposed on two opposite sides of the chamber 201. The first portion 209a is configured to transmit the ultrasonic wave and the second portion 209b is configured to receive the ultrasonic wave. For example, the first portion 209a is an ultrasonic wave transmitter or an ultrasonic wave transceiver, and the second portion 209b is an ultrasonic wave receiver or an ultrasonic wave transceiver. In some examples, the second sensor 209 is encased in metal cylinders, and then attached to the inner or outer wall of the chamber 201.

In operation, the first portion 209a of the second sensor 209 is coupled to a pulse generator (not shown) to receive a pulse of the ultrasonic wave. Then the pulse of the ultrasonic wave is transmitted by the first portion 209a. After passing through the beverage 202 in the chamber 201, the pulse of the ultrasonic wave is received by the second portion 209b of the second sensor 209, which disposed on the opposite side of the first portion 209a. The time of flight for the pulse of the ultrasonic wave is measured when a wave peak or other waveforms corresponding to the arrival of the pulse is received by the second portion 209b. The time of flight can be converted to the velocity of the ultrasonic wave by dividing the flight distance by the time of flight. It is readily appreciated that the time of flight can be determined by measuring the echo of the pulse of the ultrasonic wave that reflects between the opposite sides of the chamber 201. In some other examples, the amplitude of the pulse of the ultrasonic wave transmitted by the first portion 209a and the amplitude of the pulse of the ultrasonic wave received by the second portion 209b are measured to determine the amplitude attenuation of the ultrasonic wave during travelling through the beverage 202.

The second sensor 209 can also be an ultrasonic transceiver arranged on a wall of the chamber 201. The ultrasonic transceiver is configured to transmit and receive the ultrasonic wave. Specifically, the ultrasonic transceiver is coupled to a pulse generator (not shown) to receive a pulse of the ultrasonic wave. The pulse of the ultrasonic wave passes through the chamber 201 from one side of the chamber 201 to be reflected by the opposite side of the chamber 201, and then returns to back the side of the chamber 201 as an echo. The time of flight for the pulse of the ultrasonic wave is measured when a first wave peak is received by the second sensor 209. The time of flight can be converted to the velocity of the ultrasonic wave by dividing the flight distance, i.e. twice the diameter of the chamber 201, by the time of flight.

All these propagation characteristics of the ultrasonic wave are associated with the concentration of the predetermined substance 204 in the beverage 202. Moreover, the temperature of the beverage 202 affects the propagation characteristics of the ultrasonic wave, either. Therefore, the propagation characteristics can be used to determine the concentration of the predetermined substance 204 in combination with the temperature of the beverage 202 measured by the thermometer 211.

As depicted in FIG. 2, the apparatus 200 is integrated into a cup, which has a handle 213 extruding from the upper portion of the chamber 201. The display screen 207 is disposed on the handle 213 upward, thereby displaying the calculation result of the overall calorie to the users. In the embodiment, the apparatus 200 may further comprise a second input unit 215. The second input unit 215 is configured to receive a user instruction including the type of the predetermined substance 204 and/or the type of the beverage 202. The user instruction is further delivered to the processing unit 205. Then the processing unit 205 is further configured to calculate the overall calorie according to the type of the predetermined substance 204 and/or the type of the beverage 202. In this way, the users can input the type of the beverage or the predetermined substance to the apparatus 200 by the second input unit 215, which enables the apparatus 200 to identify different calorie-contributing substances, such as sugar or fat, thereby improving the accuracy of the calorie measurement.

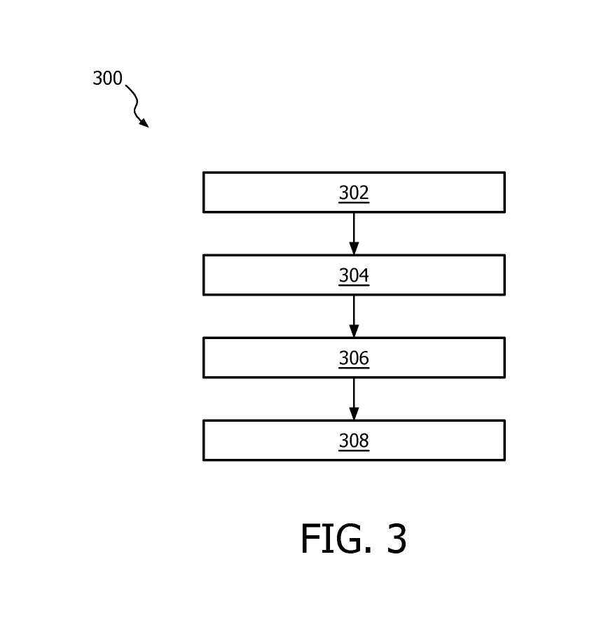

FIG. 3 depicts a method 300 for measuring calorie according to a third embodiment of the invention. The method 300 can be used to measure beverages such as tea drinks, fruit juices, energy drinks, carbonated drinks, alcoholic beverage or any other beverages containing one or more calorie-contributing substances. For example, the calorie-contributing substance may comprise one or more of sugar, alcohol, protein, fat or any other substance that contributes to the overall calorie in the beverage.

As shown in FIG. 3, the method 300 begins with obtaining beverage information indicating the volume or the weight of the beverage (Step 302). For example, the beverage information can be obtained by measuring the volume or the weight of the beverage, or by receiving an instruction including the beverage information. Then the concentration of a predetermined substance, i.e. the calorie-contributing substance in the beverage is measured (Step 304). In some examples, the concentration of the predetermined substance may be measured by a refractometer for measuring the index of refraction of the beverage. In some other examples, the concentration of the predetermined substance may be measured by measuring the propagation characteristic of the ultrasonic wave. In detail, Step 304 comprises a first step of measuring the temperature of the beverage, a second step of measuring the propagation characteristic of an ultrasonic wave passing through the beverage, and a third step of determining the concentration of the predetermined substance according to the temperature of the beverage and the propagation characteristic of the ultrasonic wave. For example, the propagation characteristic of the ultrasonic wave can be measured with an ultrasonic transceiver, which transmits the ultrasonic wave into the beverage and receives the ultrasonic wave or the echo of ultrasonic wave. Then the overall calorie in the beverage is calculated according to the volume or the weight of the beverage and the measured concentration of the predetermined substance (Step 306). Afterwards, the overall calorie calculated in Step 306 is displayed (Step 308), for example, by a display screen.

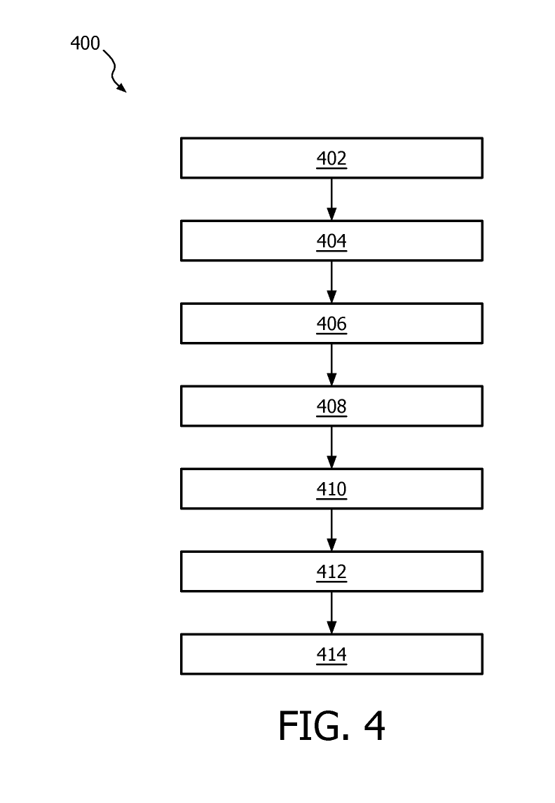

FIG. 4 depicts a method 400 for measuring calorie according to a fourth embodiment of the invention. As shown in FIG. 4, the method 400 begins with receiving a user instruction including the type of a predetermined substance and/or the type of the beverage (Step 402). User instruction can also comprise one or more other related information. The predetermined substance contributes to the overall calorie in the beverage. Then, beverage information indicating the volume or the weight of the beverage is obtained (Step 404). And the concentration of the predetermined substance is measured (Step 406). Then the overall calorie in the beverage is calculated according to the volume or the weight of the beverage, the measured concentration of the predetermined substance and the type of the predetermined substance and/or the type of the beverage (Step 408). Afterwards, the overall calorie calculated in Step 408 is displayed (Step 410), for example, by a display screen. In some examples, the method 400 may further comprises storing the results of the overall calorie (Step 412) and providing historical calorie intake information according to the results of the overall calorie (Step 414).

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single unit may fulfill the functions of several items recited in the claims. The mere fact that certain measures are recited in mutually different dependent claims does not indicate that a combination of these measured cannot be used to advantage. Any reference signs in the claims should not be construed as limiting the scope.

1. An apparatus for measuring calorie in a beverage, comprising:
a chamber, configured to contain the beverage;
an information obtaining unit, configured to obtain beverage information indicating the volume or the weight of the beverage;
a concentration measuring unit, configured to measure the concentration of a predetermined substance in the beverage;
a second sensor, configured to measure the propagation characteristic of an ultrasonic wave passing through the beverage,
a thermometer, configured to measure the temperature of the beverage;
said concentration measuring unit is further configured to determine the concentration of the predetermined substance according to the temperature of the beverage and the propagation characteristic of the ultrasonic wave;
a processing unit, configured to calculate the overall calorie according to the volume or the weight of the beverage and the measured concentration of the predetermined substance; and
a display screen, configured to display the overall calorie.

2. An apparatus as claimed in claim 1, wherein the information obtaining unit is a first sensor for measuring the volume or the weight of the beverage, or a first input unit for receiving an instruction including the beverage information.

3. (canceled)

4. An apparatus as claimed in claim 2, wherein the second sensor comprises a first portion and a second portion disposed on two opposite sides of the chamber, the first portion being configured to transmit the ultrasonic wave and the second portion being configured to receive the ultrasonic wave; or the second sensor comprises an ultrasonic transceiver arranged on a wall of the chamber, the ultrasonic transceiver being configured to transmit and receive the ultrasonic wave.

5. An apparatus as claimed in claim 1, further comprising:
a second input unit, configured to receive a user instruction including the type of the predetermined substance and/or the type of the beverage; and
the processing unit is further configured to measure the overall calorie according to the type of the predetermined substance and/or the type of the beverage.

6. An apparatus as claimed in claim 1, further comprising:
a memory, configured to store the results of the overall calorie; and
the apparatus is further configured to provide historical calorie intake information according to the results of the overall calorie.

7. A method for measuring calorie in a beverage, comprising:
obtaining beverage information indicating the volume or the weight of the beverage;
measuring the concentration of a predetermined substance in the beverage;
measuring the temperature of the beverage;
measuring the propagation characteristic of an ultrasonic wave passing through the beverage; and
determining the concentration of the predetermined substance according to the temperature of the beverage and the propagation characteristic of the ultrasonic wave;
calculating the overall calorie according to the volume or the weight of the beverage and the measured concentration of the predetermined substance; and
displaying the overall calorie.

8. (canceled)

9. A method as claimed in claim 7, wherein the step of obtaining the beverage information comprises:
obtaining the beverage information by measuring the volume or the weight of the beverage, or by receiving an instruction including the beverage information.

10. A method as claimed in claim 7, further comprising:
receiving a user instruction including the type of the predetermined substance and/or the type of the beverage; and
the calculating step further comprising calculating the overall calorie according to the type of the predetermined substance and/or the type of the beverage.
